(19) United States
(12) Patent Application Publication
(10) Pub. No.: US 2020/0138443 A1
(43) Pub. Date: May 7, 2020

(54) A SURGICAL CLAMP FOR CLAMPING AN ORGAN

(71) Applicant: Hugh Finlay MACNEIL, Gosford, New South Wales (AU)

(72) Inventor: Hugh Finlay MACNEIL, Gosford, New South Wales (AU)

(21) Appl. No.: 16/618,918

(22) PCT Filed: Jun. 5, 2017
(86) PCT No.: PCT/AU2017/050545

§ 371 Date: Dec. 3, 2019

(51) Int. Cl. A61B 17/122

(52) U.S. Cl. CPC A61B 17/122; A61B 2017/12004

(57) ABSTRACT

A surgical clamp for clamping an organ comprising: a first clamping member and a second clamping member each having a distal end and a proximal end; the first clamping member and second clamping member being connected to each other at their respective proximal ends and pivotally movable relative to each other between an open configuration and a closed configuration of the clamp; each of the first and second clamping members having one or more regions of curvature along its length; the first clamping member comprising a first tissue engaging portion having a first tissue engaging surface and the second clamping member comprising a second tissue engaging portion having a second tissue engaging surface; each of the first and second tissue engaging portions comprising an arrangement of teeth for engaging with the organ; wherein the first tissue engaging surface and the second tissue engaging surface are concave and oppose each other such that in the closed configuration, a gap is defined between the first and the second tissue engaging portions at an intermediate location along the respective first and second clamping members between their respective proximal and distal ends.

### FIELD OF THE INVENTION

The present invention relates to surgical implements and in particular to a surgical clamp for clamping an organ.

Some embodiments of the invention have been developed primarily for use in open surgery whilst others have been developed primarily for use in laparoscopic surgery. However, it will be appreciated that the invention is not limited to these particular fields of use.

### BACKGROUND OF THE INVENTION

Operations on the kidney can involve removing the whole kidney, a nephrectomy, or just the part of the kidney, a partial nephrectomy. These operations are most commonly performed for tumours of the kidney. The choice between nephrectomy and partial nephrectomy depends on complex considerations including the site and size of the tumour, reserve kidney function, other diseases present and patient preference. Minimally invasive approaches (using laparoscopic techniques, including the use of robotic laparoscopic techniques) offer the advantage of less pain after the operation and quicker return to normal activities.

However, in use of minimally invasive techniques, a larger proportion of post-operative kidney patients exhibit long-term tissue damage than with open techniques. This decline in kidney performance or renal sufficiency may only become apparent 12 months or longer after the operation.

One reason for this is because of the implements used by the surgeon to stop bleeding from the kidney during removal of tissue. The vessels to all of the kidney are usually temporarily clamped to prevent bleeding from the vessels cut within the kidney during removal of tissue such as a tumour. Thus, the rest of the kidney is deprived of blood supply for significant periods of time which may result in permanent tissue damage.

About one third of patients undergoing operations on kidney tumours already have a degree of renal insufficiency. As a result, removal of tumours of the kidneys worsens an existing problem in the aging population. There is a need for a clamp which is able to control blood loss during removal of kidney tumours without causing or at least reducing long-term detrimental effects to kidney function.

The present invention seeks to provide a surgical clamp, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

According to a first aspect of the invention, there is provided a surgical clamp for clamping an organ comprising a first clamping member and a second clamping member each having a distal end and a proximal end. The first clamping member and second clamping member being connected to each other at their respective proximal ends and pivotally movable relative to each other between an open configuration and a closed configuration of the clamp. Each of the first and second clamping members having one or more regions of curvature along its length. The first clamping member comprising a first tissue engaging portion having a first tissue engaging surface and the second clamping member comprising a second tissue engaging portion having a second tissue engaging surface. Each of the first and second tissue engaging portions comprising an arrangement of teeth for engaging with the organ. The first tissue engaging surface and the second tissue engaging surface are concave and oppose each other such that in the closed configuration, a gap is defined between the first and the second tissue engaging portions at an intermediate location along the respective first and second clamping members between their respective proximal and distal ends.

The surgical clamp assists in clamping an organ in an atraumatic manner during a surgical procedure.

In one embodiment of the invention, the organ is a kidney.

In one embodiment of the invention, the arrangement of teeth comprises a plurality of longitudinal rows of teeth.

The arrangement of teeth provides sufficient grip to prevent slipping of the clamp when applied on the organ without harming the capsule of the organ.

In one embodiment of the invention, the plurality of longitudinal rows of teeth is between 2 and 20 rows of teeth.

In one embodiment of the invention, the plurality of longitudinal rows of teeth is about 4 rows of teeth.

In one embodiment of the invention, the plurality of longitudinal rows of teeth is about 8 rows of teeth.

In one embodiment of the invention, the teeth are arranged in intersecting rows.

In one embodiment of the invention, the teeth are arranged in a cross-hatched configuration.

In one embodiment of the invention, each tooth of the arrangement of teeth is substantially a 4-sided pyramid in shape.

In one embodiment of the invention, each tooth of the arrangement of teeth is substantially a tetrahedron in shape.

In one embodiment of the invention, the width of each of the first and second clamping members is within the range of 4 mm to 25 mm.

The width of each of the first and second clamping members helps to occlude arteries within the organ without cutting or traumatizing the organ and thus, prevents excessive bleeding. Also, it is used to grip the organ to allow positioning of the organ for removal of any tumours and suturing of the organ.

In one embodiment of the invention, the width of each of the first and second clamping members is about 7 mm.

This width of each of the first and second clamping members helps to occlude arteries within the organ without cutting or traumatizing the organ. The width is narrow enough to be inserted through a laparoscopic port.

In one embodiment of the invention, the width of each of the first and second clamping members is about 12 mm.

In one embodiment of the invention, the surgical clamp further comprises a first handle and a second handle and wherein the first and second clamping members are pivotally movable by the first and second handles, respectively.

In one embodiment of the invention, each of the first and second handles is between 50 mm and 180 mm long.

In one embodiment of the invention, each of the first and second handles is about 150 mm long.

Each of the first and second handles having a length of 150 mm allows sufficient haptic feedback for safe occlusion of arteries in underlying organ without breach of the organ. This length is short enough to enable sufficient opening of the first and second clamping members to position the surgical clamp onto the organ without harming the organ.

In one embodiment of the invention, each of the first and second handles is about 180 mm long.

Each of the first and second handles having a length of 180 mm allows sufficient haptic feedback for safe occlusion of arteries in underlying organ without breach of the organ. This length is short enough to enable sufficient opening of the first and second clamping members to position the surgical clamp onto the organ.

In one embodiment of the invention, each of the first and second handles is about 80 mm long.

Each of the first and second handles having a length of about 80 mm allows sufficient adjustment of tension of the first and second clamping members to occlude arteries without injuring the organ. This length is short enough to enable manipulation of the organ laparoscopically to enable resection of tumour and suture of the organ and to avoid prolonged occlusion of the main artery to the organ.

In one embodiment of the invention, the longitudinal cross-sectional shape of each of the tissue engaging portions is substantially a first arc having a radius of curvature within the range of 90 mm to 210 mm.

In one embodiment of the invention, the first arc has a radius of curvature of about 160 mm.

The radius of curvature of the first arc is required to conform to the shape of the organ and provides occlusive pressure on the arteries in the organ in multiple different positions within the organ, without slipping off the organ.

In one embodiment of the invention, the first arc has a radius of curvature of about 104 mm.

This radius of curvature of the first arc enables occlusion of arteries in different sized organs having tumours in more peripheral regions.

In one embodiment of the invention, the first arc has a radius of curvature of about 184 mm.

The radius of curvature of the first arc is required to conform to the shape of the organ and provides occlusive pressure on the arteries in the organ in multiple different positions within the organ, without slipping off the organ. The radius of curvature of the first arc is shallow enough to allow insertion through a laparoscopic port.

In one embodiment of the invention, the length of each of the first and second clamping members is within the range of 60 mm to 120 mm.

This range of the length of each of the first and second clamping members is required to encompass any cross-section of the entire organ and enable occlusion of contained arteries.

Also, this length allows enclosure of an organ containing a tumour in a central portion, without occluding the arteries to the rest of the organ.

In one embodiment of the invention, the length of each of the first and second clamping members is about 100 mm.

In one embodiment of the invention, the length of each of the first and second clamping members is about 80 mm.

In one embodiment of the invention, the length of each of the first and second clamping members is about 85 mm.

In one embodiment of the invention, the thickness of each of the first and second clamping members is within the range of 1 to 4 mm.

The thickness of each of the first and second clamping members provides rigidity to the surgical clamp and allows occlusion of the arteries under the first and second tissue engaging portions of the first and second clamping members, respectively at all points including the distal tips of the first and second clamping members without creating a bulky unwieldy instrument and therefore, the surgical clamp is able to be inserted into narrow spaces.

In one embodiment of the invention, the thickness of each of the first and second clamping members is about 3 mm.

In one embodiment of the invention, the thickness of each of the first and second clamping members is about 2 mm.

In one embodiment of the invention, the first and second clamping members are configured such that the maximum gap defined between the two tissue engaging surfaces is between 5 mm and 29 mm when the clamp is in the closed configuration.

The two tissue engaging surfaces enable occlusion of arteries in different sized organs with the maximum gap of 5 mm to 29 mm between them.

In one embodiment of the invention, the first and second clamping members are configured such that the maximum gap defined between the two tissue engaging surfaces is between 7 mm to 16 mm when the clamp is in the closed configuration.

In one embodiment of the invention, the first and second clamping members are configured such that the maximum gap defined between the two tissue engaging surfaces is about 13 mm when the clamp is in the closed configuration.

The two tissue engaging surfaces enable occlusion of arteries in the organs with the maximum gap of 13 mm between them. Further, the closed configuration of the surgical clamp with this maximum gap allows a low enough profile of the closed clamp to insert through different sized laparoscopic ports.

The maximum gap between the first and second clamping members enables insertion through different sized laparoscopic ports with different thickness of the first and second clamping members for use on tumours at different positions in the organs.

In one embodiment of the invention, the surgical clamp has a maximum length of between 200 mm and 300 mm.

The total length of the surgical clamp is provided such that it allows balance of the two clamping members and two handles with sufficient haptic feedback for safe occlusion of the arteries in the underlying organ without injuring the organ.

In one embodiment of the invention, the surgical clamp has a maximum length of about 250 mm.

In one embodiment of the invention, each of the first and second clamping members comprises a proximal outwardly curved region and a distal inwardly curved region and the first and second tissue engaging portions are located between their respective proximal outwardly curved region and distal inwardly curved region and comprise a longitudinal cross-sectional shape that is substantially linear.

In one embodiment of the invention, the first and the second tissue engaging portions generally extend at a proximal angle of between 5 and 45 degrees, with respect to their respective proximal outwardly curved regions.

In one embodiment of the invention, the proximal angle is about 25 degrees.

In one embodiment of the invention, the proximal angle is provided by a proximal curved portion having a radius of arc of between 40 mm and 200 mm.

The radius of arc in this range enables occlusion of arteries in different sized organs and encompasses tumours in the central region of the organ.

In one embodiment of the invention, the proximal angle is provided by a proximal curved portion having a radius of arc of about 160 mm.

In one embodiment of the invention, the first and second tissue engaging portions generally extends at a distal angle of between 5 and 45 degrees, with respect to their respective distal inwardly curved regions, when the surgical clamp is in the closed configuration.

In one embodiment of the invention, the distal angle is about 25 degrees.

In one embodiment of the invention, the distal angle is provided by a distal curved portion having a radius of arc of between 40 mm and 200 mm.

The radius of arc in this range enables occlusion of arteries in different sized organs and encompasses tumours in the central region of the organ.

In one embodiment of the invention, the distal angle is provided by a distal curved portion having a radius of arc of about 160 mm.

In one embodiment of the invention, the first clamping member and the second clamping are pivotally movable relative to each other around a pivot axis and the distance between the pivot axis at a location where the first and second clamping members connect and a distal tip of each of the distal inwardly curved regions is a pivot-to-tip distance and the pivot-to-tip distance is between 70 mm and 180 mm.

The pivot-to-tip distance in the surgical clamp enables occlusion of the arteries in different sized organs such as kidneys and encompasses both peripheral and centrally located tumours in kidneys.

In one embodiment of the invention, the pivot-to-tip distance is about 160 mm.

In one embodiment of the invention, the proximal outwardly curved region and the distal inwardly curved region are between 40 mm and 90 mm in length.

The length of the proximal outwardly curved region and the distal inwardly curved region allows the surgical clamp to encompass different sized tumours in different positions in the central portion of the organ, such as, closer to the hilum in the central position of the kidney.

In one embodiment of the invention, the proximal outwardly curved region and the distal inwardly curved region are about 61 mm in length.

In one embodiment of the invention, the length of each of the first and second tissue engaging portions is between 50 mm and 100 mm.

The length of each of the first and second tissue engaging portions helps in encompassing any given cross-section of the organ containing the tumour in a more central region without occluding the arteries to the rest of the organ.

In one embodiment of the invention, the length of each of the first and second tissue engaging portions is about 80 mm.

In one embodiment of the invention, the surgical clamp has a maximum diameter in any cross-section of 18 mm.

The surgical clamp with maximum diameter of any cross-section of the clamp in the closed configuration enables insertion of the clamp through a variety of laparoscopic ports. Larger laparoscopic ports enable insertion of the clamp with a wider gap between the two tissue engaging surfaces for larger organs or more centrally located tumours.

In one embodiment of the invention, the surgical clamp has a maximum diameter in any cross-section of 13 mm.

In one embodiment of the invention, the first clamping member comprises a first proximal outwardly curved region and a first distal inwardly curved region and the first tissue engaging portion is located between the first proximal outwardly curved region and the first distal inwardly curved region and comprise a longitudinal cross-sectional shape that is linear or slightly curved and the second clamping member comprises a second proximal outwardly curved region and a second distal inwardly curved region and the second tissue engaging portion is located between the second proximal outwardly curved region and the second distal inwardly curved region and comprise a longitudinal cross-sectional shape that is linear or slightly curved.

In one embodiment of the invention, the first proximal outwardly curved region, the first distal inwardly curved region, the second proximal outwardly curved region and the second distal inwardly curved region are substantially arcuate and have a radius of between 5 mm and 30 mm.

The radius of the first proximal outwardly curved region, the first distal inwardly curved region, the second proximal outwardly curved region and the second distal inwardly curved region enables the clamp to provide occlusive pressure on the arteries in multiple different positions of the organ without slipping off the organ. This also assists in hooking the organ in place to prevent the organ sliding forwards in the clamp.

In one embodiment of the invention, the first proximal outwardly curved region, the first distal inwardly curved region, the second proximal outwardly curved region and the second distal inwardly curved region are substantially arcuate and have a radius of about 10 mm.

In one embodiment of the invention, the length of the first clamping member is less than the length of the second clamping member such that the second distal inwardly curved region is offset from the first distal inwardly curved region when the clamp is in the closed configuration.

The length of the shorter and longer clamping members allows a narrower separation of mid-regions of the shorter and longer clamping members providing for insertion through narrower laparoscopic ports.

In one embodiment of the invention, the length of the first clamping member is between 60 mm and 90 mm.

In one embodiment of the invention, the length of the first clamping member is about 80 mm.

In one embodiment of the invention, the length of the second clamping member is between 70 and 100 mm.

In one embodiment of the invention, the length of the second clamping member is about 90 mm.

In one embodiment of the invention, the distance between the first distal inwardly curved region and the second distal inwardly curved region is an offset distance of between 5 mm and 25 mm.

The offset distance between the first distal inwardly curved region and the second distal inwardly curved region allows a lower profile of the clamp while preserving separation of the two tissue engaging portions when closed for insertion through a smaller laparoscopic port.

In one embodiment of the invention, the offset distance is about 10 mm.

In one embodiment of the invention, the first and second clamping members fall within a maximum diameter of 17 mm when the clamp is in the closed configuration.

The maximum diameter of the first and second clamping members allows insertion of the clamp through a variety of laparoscopic ports.

In one embodiment of the invention, the first and second clamping members fall within a maximum diameter of 13 mm when the clamp is in the closed configuration.

In one embodiment of the invention, the first and second clamping members fall within a maximum diameter of 10 mm when the clamp is in the closed configuration.

In one embodiment of the invention, the proximal ends of the first and second clamping members are configured for connection to a surgical robot.

The first and second clamping members connected to the surgical robot may be used in robotically assisted surgery.

In one embodiment of the invention, the proximal ends of the first and second clamping members are connected by a pivot mechanism.

In one embodiment of the invention, the pivot mechanism is an axle.

In one embodiment of the invention, the surgical clamp further comprises a first and second lever member and wherein the first clamping member is integral with the first lever member and the second clamping member is integral with the second lever member such that relative movement of the first and second lever members towards each other causes the first and second clamping members to move apart and relative movement of the first and second lever members away from each other causes the first and second clamping members to move toward each other.

In one embodiment of the invention, the first lever member is a first handle and the second lever member is a second handle.

In one embodiment of the invention, the surgical clamp further comprises an actuator for driving the first and second clamping members.

The actuator may be located at a significant distance from the surgical clamp end and used to manipulate the surgical clamp during surgery. Further, the actuator may comprise a variety of different manipulators.

In one embodiment of the invention, the actuator is a scissor-jack actuation arrangement.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

Other aspects of the invention are also disclosed.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 shows a perspective view of a surgical clamp in accordance with a first embodiment of the present invention in a closed position;

FIG. 2 shows a perspective view of the first embodiment in a partially open position;

FIG. 3 shows an enlarged view of a pivoting arrangement of some embodiments of a surgical clamp in accordance with the present invention;

FIG. 4 shows an enlarged view of a tissue engaging portion of some embodiments of a surgical clamp in accordance with the present invention where teeth of a tissue engaging portion are arranged in parallel, longitudinal rows spaced from each other across the width of the tissue engaging portion;

FIG. 5 shows an enlarged view of a tissue engaging portion of some embodiments of a surgical clamp in accordance with the present invention where teeth of the tissue engaging portion are arranged in a cross hatched formation;

FIG. 6 shows a rearview of a surgical clamp in accordance with a third embodiment of the present invention in a closed position;

FIG. 7 shows an enlarged view of a portion of a tissue engaging portion of a surgical clamp in accordance with the present invention wherein the teeth are arranged in intersecting rows;

FIG. 8 shows a perspective view of the third embodiment of the surgical clamp in a partially closed position;

FIG. 9 shows a view from behind of the third embodiment of the surgical clamp in the closed position;

FIG. 10 shows a view from the above of the third embodiment of the surgical clamp

FIG. 11 shows a side view of a surgical clamp in accordance with a fourth embodiment of the present invention in a closed position;

FIG. 12 shows an enlarged view of a portion of a tissue engaging portion of a surgical clamp of the fourth embodiment wherein the teeth are arranged in intersecting rows;

FIG. 13 shows a perspective view of the surgical clamp of the fourth embodiment in an open position;

FIG. 14 shows a surgical clamp in of a fifth embodiment of the present invention having one clamping member that is longer than the other clamping member, in a closed position;

FIG. 15 shows an enlarged view of a portion of a tissue engaging portion of a surgical clamp of the fifth embodiment wherein the teeth are arranged in intersecting rows;

FIG. 16 shows a perspective view of the surgical clamp of the fifth embodiment in an open position;

FIG. 17 shows a side view of a surgical clamp in accordance with a sixth embodiment of the present invention in a closed position;

FIG. 18 shows the side view of the surgical clamp in accordance with the sixth embodiment of the present invention in a fully open configuration;

FIG. 19 shows an enlarged view of a portion of a tissue engaging portion of a surgical clamp of the sixth embodiment wherein the teeth are arranged in parallel, longitudinal rows spaced from each other across the width of the tissue engaging portion;

FIG. 20 shows an enlarged view of a lever of the surgical clamp in accordance with the sixth embodiment of the present invention; and

FIG. 21 shows a perspective view of the surgical clamp of the sixth embodiment in an open position.

### DESCRIPTION OF EMBODIMENTS

It should be noted in the following description that like or the same reference numerals in different embodiments denote the same or similar features.

A surgical clamp for clamping an organ according to a first aspect of the invention is generally indicated by the numeral 100.

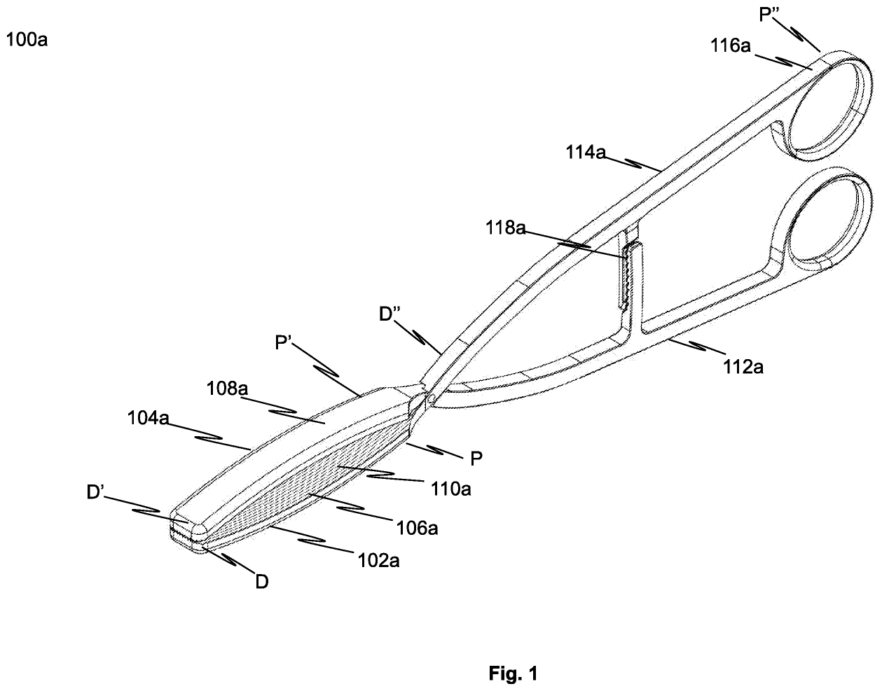

In one embodiment now described, the surgical clamp (100a) as shown in FIG. 1, comprises a first clamping member (102a), a second clamping member (104a), a first tissue engaging portion (106a), a second tissue engaging portion (108). Each of the first tissue engaging portion (106a) and the second tissue engaging portion (108a) comprise an arrangement of teeth (110a).

The first clamping member (102a) has a distal end (D) and a proximal end (P) and it may extend continuously from the proximal end (P) to the distal end (D). The first clamping member (102a) comprises a first tissue engaging portion (106a) and the first tissue engaging portion (106a) provides the arrangement of teeth (110a) for engaging with the surface of the organ. Further, the first clamping member (102a) has one or more regions of curvature along its length.

The second clamping member (104a) has a distal end (D′) and a proximal end (P′) and it may extend continuously from the proximal end (P) to the distal end (D′). The second clamping member (104a) comprises a second tissue engaging portion (108a) and the second tissue engaging portion (108a) provides the arrangement of teeth (110a) for engaging with the surface of the organ. Further, the second clamping member (104a) has one or more regions of curvature along its length.

The first tissue engaging portion (106a) has a first tissue engaging surface and the second tissue engaging portion (108a) has a second tissue engaging surface. The first tissue engaging surface and the second tissue engaging surface are generally concave and oppose each other such that a gap is formed between the two tissue engaging surfaces for a significant proportion of the overall length of the first and second clamping members (102a, 104a) when the surgical clamp (100a) is in a closed configuration. The first tissue engaging portion (106a) and the second tissue engaging portion (108a) extend over a substantial length of the first clamping member (102a) and the second clamping member (104a), respectively. The longitudinal cross-sectional shape of the first tissue engaging portion (106a) and the second tissue engaging portion (108a) is substantially a first arc having a radius of curvature within the range of 90 mm to 210 mm. Preferably, the first arc has a radius of curvature of about 160 mm.

The first tissue engaging surface and the second tissue engaging surface of the respective first tissue engaging portion (106a) and the second tissue engaging portion (108a) holds the organ when clamped. The surgical clamp is specifically designed for clamping an organ such as a kidney. The first clamping member (102a) and the second clamping member (104a) are configured such that the maximum gap formed between the two tissue engaging surfaces is between 7 mm to 29 mm when the surgical clamp (100a) is in the closed configuration. Preferably, the maximum gap formed between the two tissue engaging surfaces is about 13 mm when the surgical clamp (100a) is in the closed configuration.

Each of the first clamping member (102a) and the second clamping member (104a) has a width in the range of 4 mm to 25 mm. Preferably, the first clamping member (102a) and the second clamping member (104a) has a width of about 7 mm. The length of each of the first clamping member (102a) and the second clamping member (104a) is in the range of 80 mm to 120 mm. Preferably, the length of each of the first clamping member (102a) and the second clamping member (104a) is about 100 mm. Further, the thickness of each of the first clamping member (102a) and the second clamping member (104a) is in the range of 1 mm to 4 mm. Preferably, the thickness of each of the first clamping member (102a) and the second clamping member (104a) is about 3 mm.

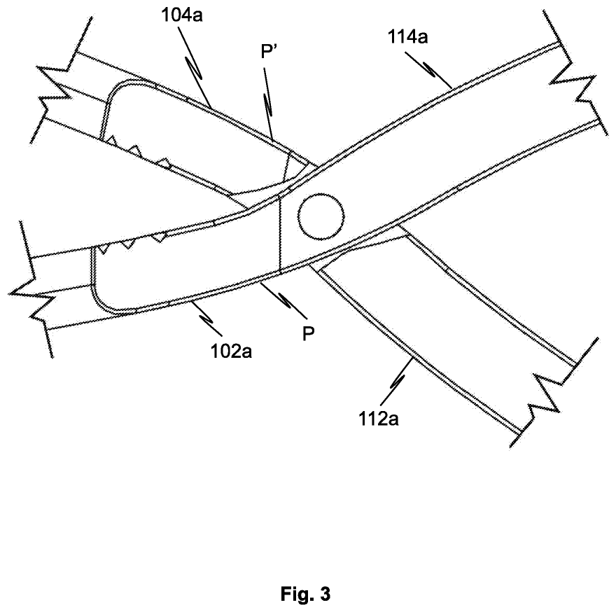

Referring to FIG. 3, a pivoting arrangement of the first clamping member (102a) and the second clamping member (104a) is provided. The first clamping member (102a) and the second clamping member (104a) are connected to each other at their respective proximal ends (P) and (P′) and pivotally movable relative to each other between an open configuration and closed configuration of the surgical clamp (100a). The first tissue engaging portion (106a) and the second tissue engaging portion (108a) engages with the organ by the arrangement of teeth (110a) such that in the closed configuration the maximum gap is defined between the first tissue engaging portion (106a) and the second tissue engaging portion (108a) at an intermediate location along the respective first clamping member (102a) and the second clamping member (104a) between their respective proximal ends (P), (P′) and distal ends (D), (D′).

The surgical clamp (100a) further comprises a first handle (112a) and a second handle (114a). Each of the first handle (112a) and the second handle (114a) has a thumb/finger engaging aperture (116a) at a proximal end (P″) for securely holding the surgical clamp (100a). The first clamping member (102a) and the second clamping member (104a) are pivotally movable by the first handle (112a) and the second handle (114a), respectively. Each of the first handle (112a) and the second handle (114a) has a length in the range of 130 mm to 180 mm. The length of each of the first handle (112a) and the second handle (114a) is preferably, 150 mm. The surgical clamp (100a) has a total length between 200 mm and 300 mm. Preferably, the surgical clamp (100a) has a total length of about 250 mm.

The surgical clamp (100a) comprises a ratchet (118a) such that it is positioned substantially near a distal end (D″) of the first handle (112a) and the second handle (114a). The ratchet (118a) provided on the surgical clamp (100a) is suitable for a left-handed user. In other embodiments (not shown), the ratchet (118) is also provided the other way around such that it can be easily operated by a right-handed user.

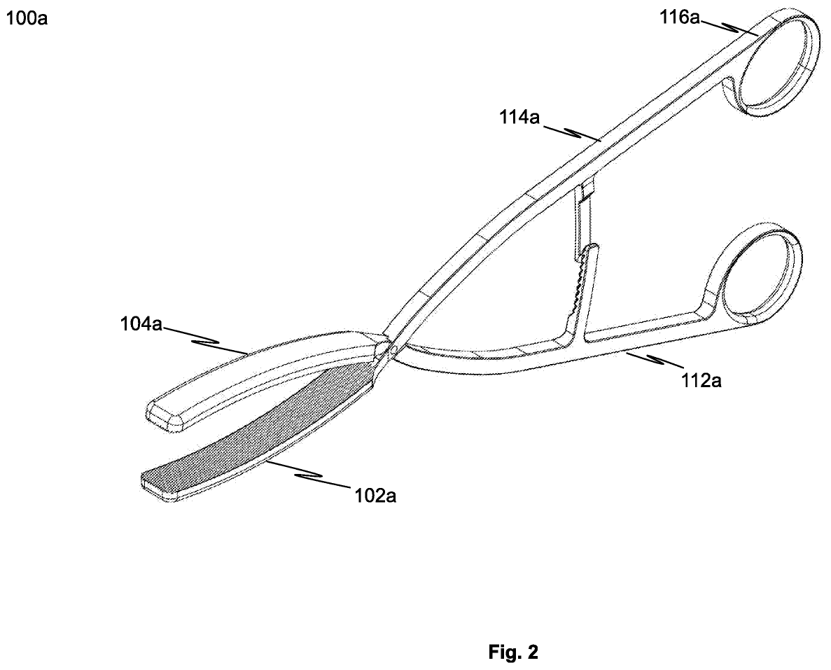

To position the surgical clamp (100a) within a surgical site or onto the organ such as kidney, the surgical clamp (100a) is brought into the open configuration as shown in FIG. 2, by using the first handle (112a) and the second handle (114a) such that the first clamping member (102a) and the second clamping member (104a) move apart from each other. In the closed configuration, the two tissue engaging portions (106a), (108a) of the respective two clamping members (102a), (104a) engages with the organ and interrupts the blood flow only in the portion of the organ containing the tumour such as in case of partial nephrectomy for kidney cancer, without injuring the organ as well as enables careful suturing of the organ to stop the bleeding before the surgical clamp (100a) is released from the surgical site.

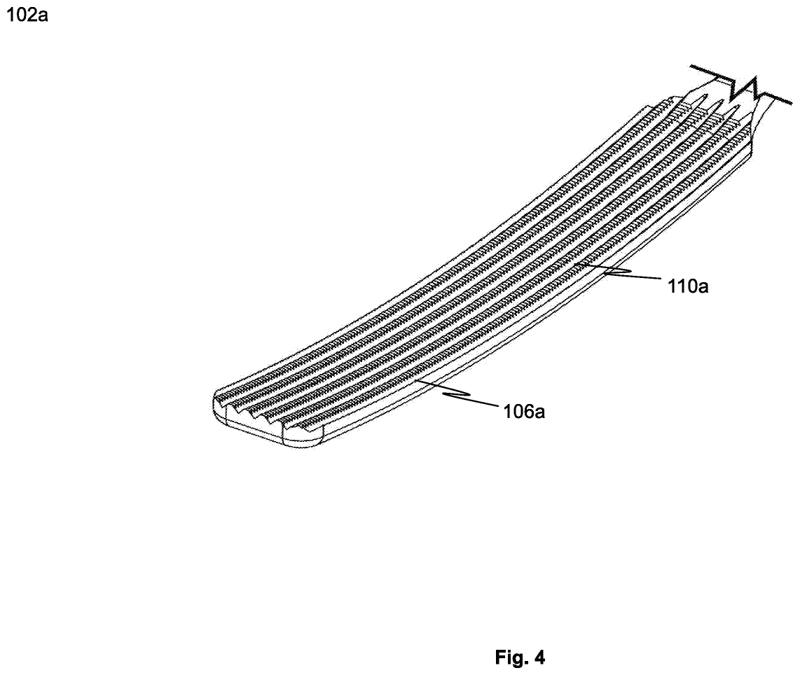

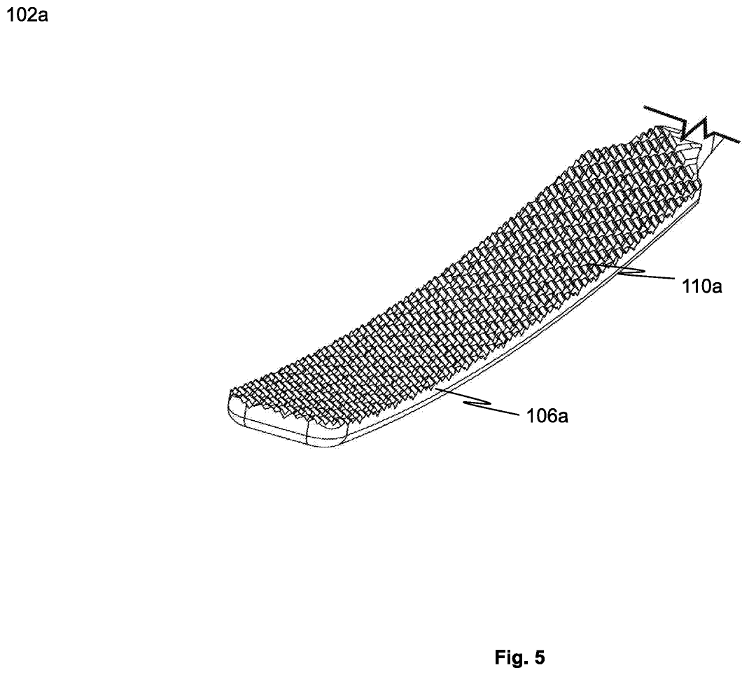

Referring to FIGS. 4 and 5, the arrangement of teeth (110a) comprises a plurality of longitudinal rows of teeth. The plurality of longitudinal rows of teeth could be between 2 and 20 rows. Preferably, the plurality of longitudinal rows of teeth is 4 rows of teeth. Further, the teeth can be arranged in intersecting rows. As shown in FIG. 4, the first tissue engaging portion (106a) and the second tissue engaging portion (108a) of the respective first clamping member (102a) and the second clamping member (104a) have the arrangement of teeth (110a) in longitudinal rows spaced from each other across the width of the two clamping members (102a), (104a). The first tissue engaging portion (106a) and the second tissue engaging portion (108a) preferably, have the arrangement of teeth (110a) in a cross hatched formation, as shown in FIG. 5.

Each tooth of the arrangement of teeth (110a) is substantially a 4-sided pyramid or a tetrahedron or other similar 3-dimensional shape. Each tooth has a maximum height in the range of 0.5 mm to 1.5 mm. Further, each tooth may be shaped as a polyhedron with a flat and/or blunted top surface for atraumatic engagement with the organ. The arrangement of teeth (110a) enables the surgical clamp (100a) to grip the organ and prevent the surgical clamp (100a) from slipping without cutting or traumatizing the underlying organ.

The first tissue engaging surface and the second tissue engaging surface of the first tissue engaging portion (106a) and the second tissue engaging portion (108a), respectively, may be composed of a resilient material. The teeth may be covered with a layer of the resilient material or the teeth themselves may be made up of the resilient material. The layer of the resilient material may be interposed between the teeth and the two tissue engaging portions (106a), (108a). In other embodiments, the whole of the surgical clamp is made of a metal such as medical grade stainless steel.

The surgical clamp (100a) may be used during open operations, such as open partial nephrectomy. The surgical clamp (100a) is used to clamp either end of the organ such as a kidney having a tumour to enable safe removal of tumours in more central regions of the kidney than the second embodiment, without cutting blood supply to the rest of the kidney and thereby reducing damaging to long term renal function.

In a second embodiment of the invention, the surgical clamp (100b) comprises the first clamping member (102b), the second clamping member (104b), the first tissue engaging portion (106b), the second tissue engaging portion (108b), the arrangement of teeth (110b), the first handle (112b) and the second handle (114b).

Each of the components of the surgical clamp (100b) of the second embodiment and their arrangement in the surgical clamp (100b) is the same as the components and their arrangement of the first embodiment of the surgical clamp (100a). Dimensions of each of the components of the surgical clamp (100b) of the second embodiment are set out below.

Each of the first handle (112b) and the second handle (114b) has a length in the range of 130 mm to 180 mm. The length of each of the first handle (112b) and the second handle (114b) is preferably, 150 mm.

The radius of arc of the first clamping member (102b) and the second clamping member (104b) is within the range of 90 mm to 210 mm. Preferably, the radius of arc of the first clamping member (102b) and the second clamping member (104b) is about 104 mm.

Each of the first clamping member (102b) and the second clamping member (104b) has a width in the range of 4 mm to 25 mm. Preferably, the first clamping member (102b) and the second clamping member (104b) has a width of about 12 mm. The length of each of the first clamping member (102b) and the second clamping member (104b) is in the range of 60 mm to 100 mm. Preferably, the length of each of the first clamping member (102b) and the second clamping member (104b) is about 80 mm. Further, the thickness of each of the first clamping member (102b) and the second clamping member (104b) is in the range of 1 mm to 4 mm. Preferably, the thickness of each of the first clamping member (102b) and the second clamping member (104b) is about 3 mm.

In the closed configuration of the surgical clamp (100b), the maximum gap formed between the two tissue engaging surfaces of the two tissue engaging portions (106b), (108b) is between 7 mm to 29 mm. Preferably, the maximum gap formed between the two tissue engaging surfaces is 13 mm. The arrangement of teeth (110b) of the two tissue engaging portions (106b), (108b) has the plurality of longitudinal rows of teeth between 2 and 20. Preferably, the plurality of longitudinal rows of teeth is 8.

The surgical clamp (100b) may be used during open operations, such as open partial nephrectomy. The surgical clamp (100b) is used to clamp either end of the organ such as kidney having a tumour to enable safe removal of tumours in common positions without cutting blood supply to the rest of the kidney and thereby damaging overall renal function for long term. The surgical clamp (100b) allows better and safer grip for manoeuvring the kidney than the first embodiment.

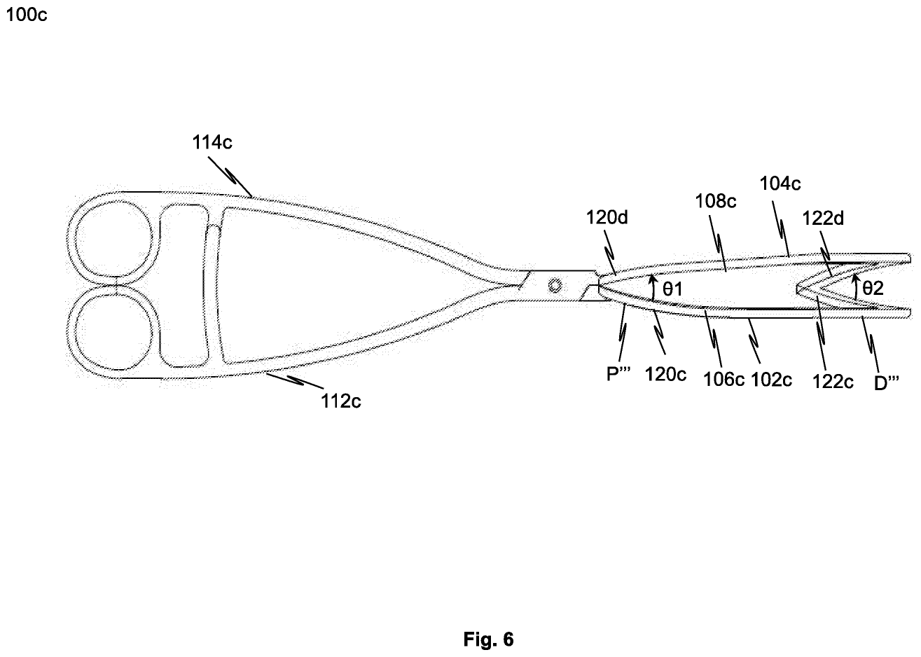

In a third embodiment of the invention, the surgical clamp (100c) as shown in FIG. 6, comprises a first clamping member (102c), a second clamping member (104c), a first tissue engaging portion (106c), a second tissue engaging portion (108c). Each of the first and second tissue engaging portions (106c, 108c) comprise an arrangement of teeth (110c).

Each of the first clamping member (102c) and the second clamping member (104c) comprises a proximal outwardly curved region (120c) at a proximal end (P″) and a distal inwardly curved region (122c) at a distal end (D″) and the first and second tissue engaging portions (106c), (108c) are located between their respective proximal outwardly curved region (120c) and distal inwardly curved region (122c). Further, each of the first tissue engaging portion (106c) and the second tissue engaging portion (108c) comprise a longitudinal cross-sectional shape that is substantially flattened.

The first tissue engaging portion (106c) and the second tissue engaging portion (108c) generally extend at a proximal angle (::1) of between 5 and 45 degrees, with respect to their respective proximal outwardly curved regions (120c). Preferably, the proximal angle (::1) is about 25 degrees. The proximal angle is provided by a proximal curved portion (120d) of the proximal outwardly curved region (120c) having a radius of arc in the range of 40 mm and 200 mm. Preferably, the radius of the proximal curved portion (120d) of the proximal outwardly curved region (120c) is about 160 mm.

The first tissue engaging portion (106c) and the second tissue engaging portion (108c) generally extend at a distal angle (::2) of between 5 and 45 degrees, with respect to their respective distal inwardly curved regions (122c), when the surgical clamp (100c) is in the closed configuration. Preferably, the distal angle (::2) is about 25 degrees. The distal angle (::2) is provided by a distal curved portion (122d) of the distal inwardly curved region (122c) having a radius of arc in the range of 40 mm and 200 mm. Preferably, the distal curved portion (122d) of the distal inwardly curved region (122c) has a radius of about 160 mm.

Each of the proximal outwardly curved regions (120c) and the distal inwardly curved regions (122c) has a length of between 40 mm and 90 mm. Furthermore, the proximal outwardly curved region (120c) and the distal inwardly curved region (122c) each have a length of about 61 mm. The length of each of the first tissue engaging portion (106c) and the second tissue engaging portion (108c) is between 50 mm and 100 mm. Preferably, the length of each of the first tissue engaging portion (106c) and the second tissue engaging portion (108c) is about 80 mm.

The first clamping member (102c) and the second clamping (104c) are pivotally movable relative to each other around a pivot axis and the distance between the pivot axis at a location where the first clamping member (102c) and the second clamping member (104c) connect and a distal tip (D″) of each of the distal inwardly curved regions (122c) is a pivot-to-tip distance and the pivot-to-tip distance is between 70 mm and 180 mm. Preferably, the pivot-to-tip distance is about 160 mm.

The pivot axis extends substantially perpendicularly to the longitudinal axis of the proximal outwardly curved region (120c) of each of the first clamping member (102c) and the second clamping member (104c).

The first clamping member (102c) and the second clamping member (104) are configured such that the maximum gap formed between the two tissue engaging surfaces of the two tissue engaging portions (106c), (108c) is between 7 mm and 29 mm when the surgical clamp (100c) is in the closed configuration. Preferably, the maximum gap formed between the two tissue engaging surfaces is about 13 mm when the surgical clamp (100c) is in the closed configuration.

Each of the first clamping member (102c) and the second clamping member (104c) has a width in the range of 4 mm to 20 mm. Preferably, the first clamping member (102c) and the second clamping member (104c) has a width of about 7 mm. The length of each of the first clamping member (102c) and the second clamping member (104c) is in the range of 80 mm to 120 mm. Preferably, the length of each of the first clamping member (102c) and the second clamping member (104c) is about 100 mm. Further, the thickness of each of the first clamping member (102c) and the second clamping member (104c) is in the range of 1 mm to 4 mm. Preferably, the thickness of each of the first clamping member (102c) and the second clamping member (104c) is about 3 mm.

The surgical clamp (100c) further includes a first handle (112c) and a second handle (114c) wherein the first clamping member (102c) and the second clamping member (104c) are pivotally movable by the first handle (112c) and the second handle (114c), respectively. Each of the first handle (112c) and the second handle (114c) has a length in the range of 130 mm to 180 mm. The length of each of the first handle (112c) and the second handle (114c) is preferably, 180 mm. Total length of the surgical clamp (100c) is between 200 mm and 300 mm. Preferably, the total length of the surgical clamp (100c) is of about 250 mm.

In operation, the two tissue engaging portions (106c), (108c) of the respective two clamping members (102c), (104c) engage with the organ when the surgical clamp is in or near the closed configuration and interrupts the blood flow only in the portion of the organ containing the tumour such as in case of partial nephrectomy for kidney cancer, without injuring the organ as well as enables careful suturing of the organ to stop the bleeding before the surgical clamp (100c) is released from the surgical site. The interruption in the blood flow is achieved by applying enough pressure of, but not limited to, 120-200 mmHg on the kidney. Each of the proximal outwardly curved regions (120c) and the distal inwardly curved regions (122c) of the respective clamping members (102c), (104c) apply the pressure evenly on the kidney and interrupt the blood flow.

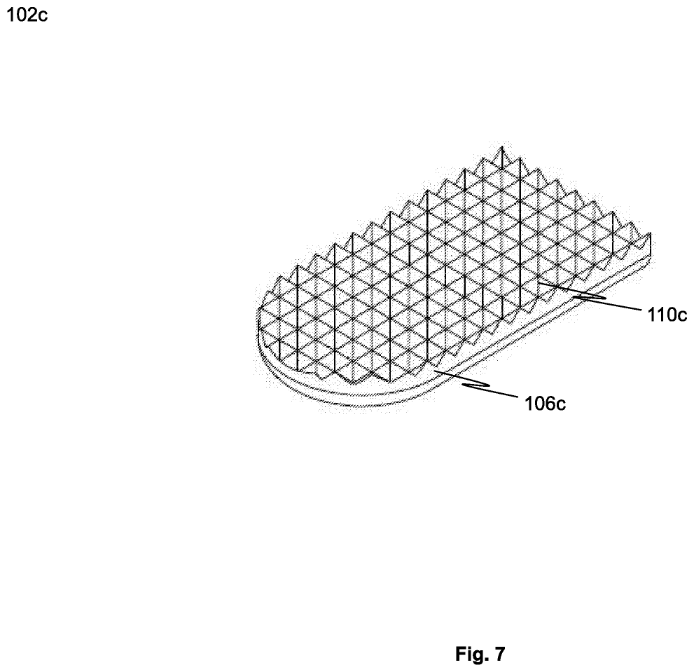

Referring to FIG. 7, the first tissue engaging portion (106c) and the second tissue engaging portion (108c) of the respective first clamping member (102c) and the second clamping member (104c) have the arrangement of teeth (110c) comprising plurality of longitudinal rows of teeth in intersecting rows. The plurality of longitudinal rows of teeth is between 2 and 20. Preferably, the plurality of longitudinal rows of teeth is 4. Each tooth of the arrangement of teeth (110c) is substantially a 4-sided pyramid or a 3-sided pyramid in shape.

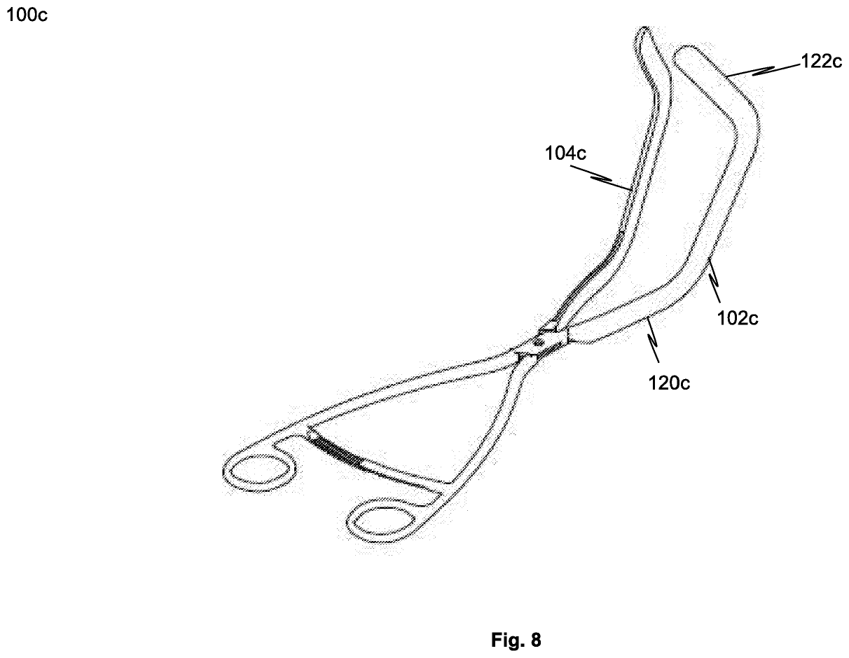

Referring to FIG. 8, perspective view of the surgical clamp (100c) in a partly closed configuration is provided. The proximal outwardly curved region (120c) of each of the first clamping member (102c) and the second clamping member (104c) has a radius of arc in the range of 40 mm to 200 mm. Preferably, the proximal outwardly curved region (120c) has a radius of arc of about 160 mm. The distal inwardly curved region (122c) of each of the first clamping member (102c) and the second clamping member (104c) has a radius of arc in the range of 40 mm to 200 mm. Further, the distal inwardly curved region (122c) has a radius of arc of about 160 mm.

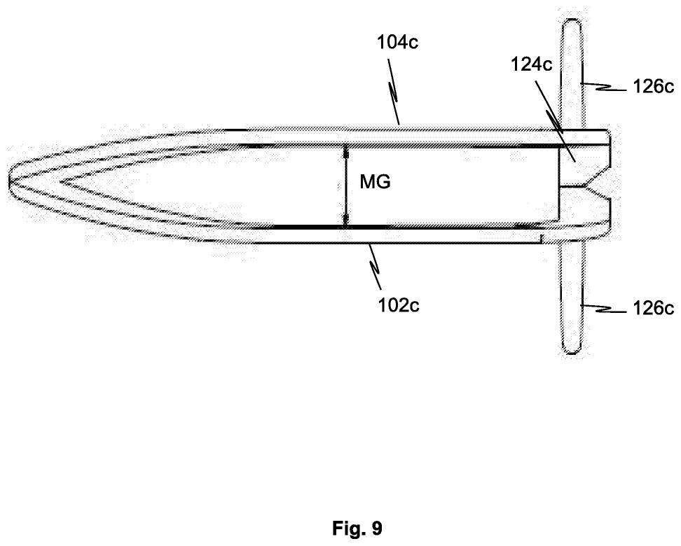

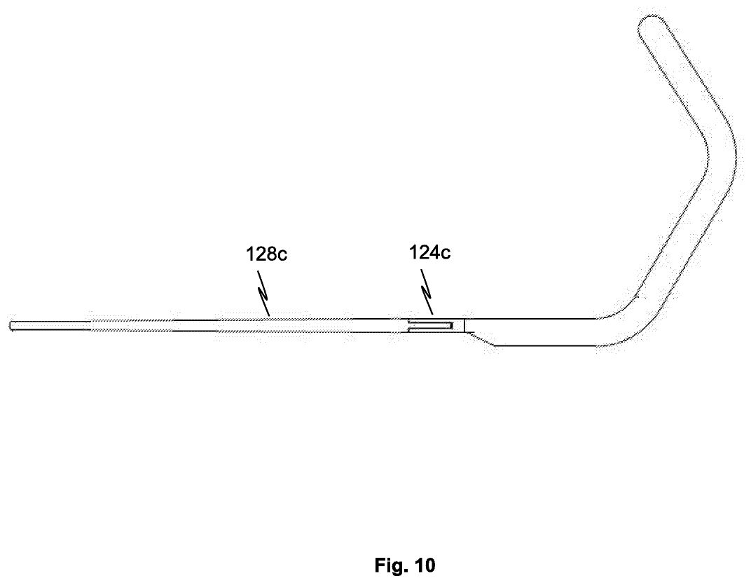

Referring to FIG. 9, the first clamping member (102c) and the second clamping member (104c) are connected to an actuation mechanism (124c) which enables movement of at least one of the first clamping member (102c) and the second clamping member (104c) relative to the other between an open configuration and a closed configuration of the surgical clamp (100c). The actuating mechanism (124c) may comprise a hinged arrangement such as is provided by a pivot axle. The actuating portion may include two tabs (126c) connected at a pivot portion wherein the first clamping member (102c) and the second clamping member (104c) move apart into the open configuration when the tabs (126c) are brought together by a user. Preferably, as shown in FIG. 10, the actuating portion may comprise an elongate extension (128c) ending in a loop, such as the thumb or finger engaging aperture (116c) for receiving a digit of hand of the user such that the surgical clamp (100c) is operable in a similar manner to scissors.

The maximum gap (MG), as shown in FIG. 9, formed between the two tissue engaging surfaces of the two tissue engaging portions (106c), (108c) of the respective clamping members (102c), (104c) is between 7 mm to 29 mm when the surgical clamp (100c) is in the closed configuration. Preferably, the maximum gap formed between the two tissue engaging surfaces is about 13 mm when the surgical clamp (100c) is in the closed configuration.

The surgical clamp (100c) may be used during open operations. The surgical clamp (100c) is used to clamp a portion of the organ such as kidney containing tumour (interpolar tumour) to enable safe removal of tumours in less common positions without cutting blood supply to the rest of the kidney and thereby damaging overall renal function in the long term.

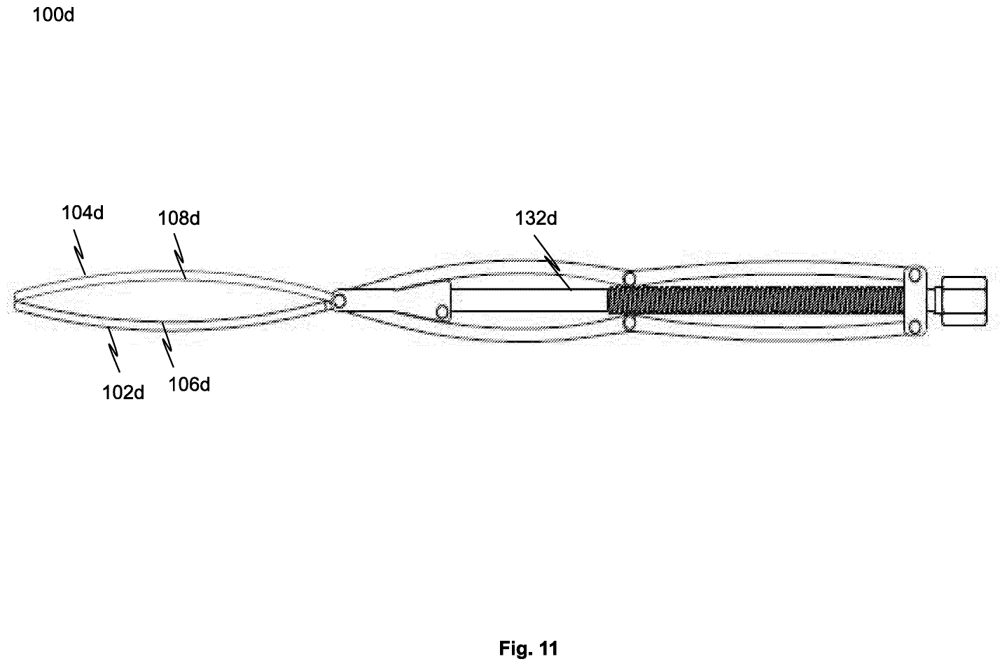

In the fourth embodiment of the invention, the surgical clamp (100d) as shown in FIG. 11, comprises a first clamping member (102d), a second clamping member (104d), a first tissue engaging portion (106d), a second tissue engaging portion (108d). Each of the first tissue engaging portion (106d) and the second tissue engaging portion (108d) comprise an arrangement of teeth (110d).

The structural arrangement of the first clamping member (102d), second clamping member (104d), first tissue engaging portion (106d), second tissue engaging portion (108d) and the arrangement of teeth (110d) in the fourth embodiment of the surgical clamp (100d) is the same as of the arrangement of these components in the first embodiment of the surgical clamp (100a). Dimensions of these components of the surgical clamp (100d) are discussed below.

Each of the first clamping member (102d) and the second clamping member (104d) has a radius of arc within the range of 150 mm to 210 mm. Preferably, the radius of arc of the first clamping member (102) and the second clamping member (104) is about 184 mm.

Each of the first clamping member (102d) and the second clamping member (104d) has a width in the range of 4 mm to 10 mm. Preferably, the first clamping member (102d) and the second clamping member (104d) has a width of about 7 mm. The length of each of the first clamping member (102d) and the second clamping member (104d) is in the range of 60 mm to 120 mm. Preferably, the length of each of the first clamping member (102d) and the second clamping member (104d) is about 85 mm. The thickness of each of the first clamping member (102d) and the second clamping member (104d) is in the range of 1 mm to 3 mm. Preferably, the thickness of each of the first clamping member (102d) and the second clamping member (104d) is about 2 mm.

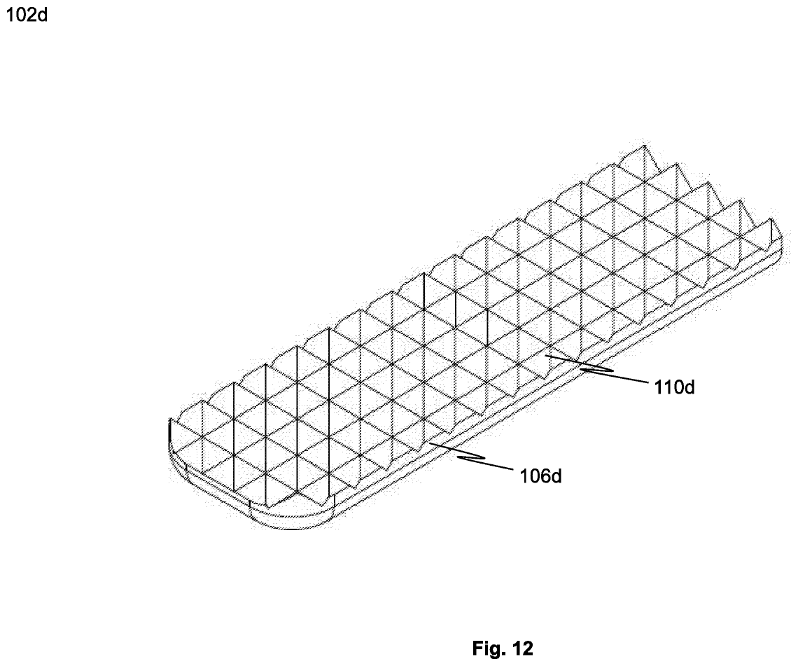

In the closed configuration of the surgical clamp (100d), the maximum gap defined between the two tissue engaging surfaces of the two tissue engaging portions (106d), (108d) of the respective clamping members (102d), (104d) is between 7 mm to 16 mm. Preferably, the maximum gap defined between the two tissue engaging surfaces is 13 mm. The arrangement of teeth (110d) (as shown in FIG. 12), of the two tissue engaging portions (106d), (108d) has the plurality of longitudinal rows of teeth in intersecting rows. The plurality of longitudinal rows of teeth is between 2 and 5 rows of teeth. Preferably, the plurality of longitudinal rows of teeth is 4. Each tooth of the arrangement of teeth (110d) is substantially a 4-sided pyramid or a 3-sided pyramid in shape.

Further, the arrangement of teeth (110d) may have any type of other suitable configuration.

In one embodiment, in the closed configuration, the surgical clamp (100d) has a maximum diameter of any cross-section of the surgical clamp (100d) in the range of 13 mm to 18 mm. In another embodiment, in the closed configuration, the maximum diameter of any cross-section of the surgical clamp (100d) is about 13 mm.

The surgical clamp (100d) further includes an actuator (132d) which enables movement of the first clamping member (102d) and the second clamping member (104d) relative to each other between the open configuration and the closed configuration of the surgical clamp (100d). The actuator (132d) may comprise a variety of manipulators (134d) such as a series of segments including sliding or jointed pieces which move the surgical clamp (100d) a number of degrees of freedom. The manipulators (134d) are pivotally connected to the first clamping member (102d) and the second clamping member (104d). Further, the actuator (132d) may comprise a scissor-jack configuration, in which the manipulators (134d) expand or contract in a manner resembling a pair of scissors upon clockwise or counter-clockwise rotation of the actuator (132d). This scissor jack has the smallest cross-sectional diameter in the closed configuration and can be opened sufficiently for the jaws to pass around the kidney without injuring the kidney. Alternatively, the actuating handles can be truncated to enable manipulation by the arms of a surgical robot, still with a scissor action at the pivot of the two elements of the clamp. Apposition of the actuating arms closes the tissue engaging portions of the clamp

Each of the manipulators (134d) has a length in the range of 50 mm to 100 mm. The length of each of the manipulators (134d) is preferably, 80 mm. Each of the manipulators (134d) may have a maximum length of 50 mm to 120 mm. The maximum length of each of the manipulators (134d) is preferably, 80 mm.

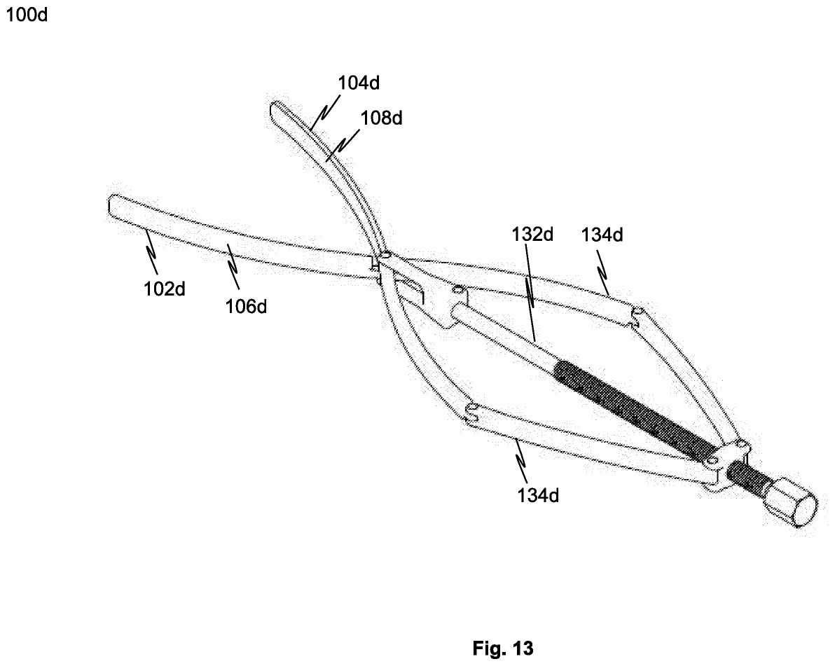

Referring to FIG. 13, the actuator (132d) is rotated in an anti-clockwise direction such that the manipulators (134d) move apart from each other. Since, the first clamping member (102d) and the second clamping member (104d) are connected pivotally with the manipulators (134d), the movement of the manipulators (134d) enables the first clamping member (102d) and the second clamping member (104d) to move away from each other for bringing the surgical clamp (100d) in the open configuration. The actuator (132d) may be located at a significant distance from the clamping members (102d), (104d) and used to manipulate the surgical clamp (100d) during surgery.

The surgical clamp (100d) may be used during a laparoscopic surgery, such as laparoscopic partial nephrectomy, as an end-effector. In the closed configuration, a maximum height of the surgical clamp (100d) may be sized to fit a laparoscopic port. Thus, the maximum height of the surgical clamp (100d) may be within the range of 11 mm to 20 mm. The surgical clamp (100d) is used to clamp either end of the organ such as kidney to enable safe removal of tumours in common positions without cutting blood supply to the rest of the kidney and thereby damaging overall renal function for long term. The surgical clamp (100d) may be used in robotically assisted surgery in which the actuator (132d) may be a part of a robotic arm as described above.

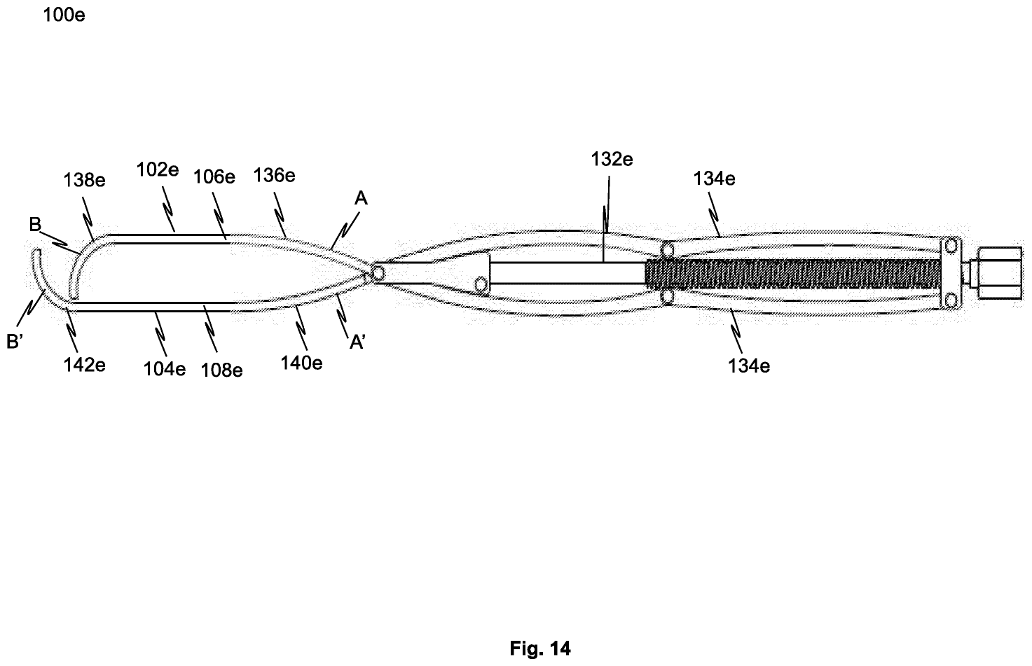

In the fifth embodiment of the invention, the surgical clamp (100e) as shown in FIG. 14, comprises a first clamping member (102e), a second clamping member (104e), a first tissue engaging portion (106e), a second tissue engaging portion (108e). Each of the first tissue engaging portion (106e) and the second tissue engaging portion (108e) comprise an arrangement of teeth (110e).

The first clamping member (102e) comprises a first proximal outwardly curved region (136e) at a proximal end (A) and a first distal inwardly curved region (138e) at a distal end (B) and the first tissue engaging portion (106e) is located between the first proximal outwardly curved region (136e) and the first distal inwardly curved region (138e) and comprise a longitudinal cross-sectional shape that is flattened or slightly curved. The second clamping member (104e) comprises a second proximal outwardly curved region (140e) at a proximal end (A) and a second distal inwardly curved region (142e) at a distal end (B) and the second tissue engaging portion (108e) is located between the second proximal outwardly curved region (140e) and the second distal inwardly curved region (142e) and comprise a longitudinal cross-sectional shape that is flattened or slightly curved.

The first proximal outwardly curved region (136e), the first distal inwardly curved region (138e), the second proximal outwardly curved region (140e) and the second distal inwardly curved region (142e) are substantially arcuate and have a radius of arc of between 5 mm and 30 mm. Preferably, the radius of arc is about 10 mm.

The length of the first clamping member (102e) is less than the length of the second clamping member (104e) such that the second distal inwardly curved region (142e) is offset from the first distal inwardly curved region (138e) when the surgical clamp (100e) is in the closed configuration. The length of the first clamping member (100e) is between 60 mm and 90 mm. Preferably, the length of the first clamping member (102e) is about 80 mm. The length of the second clamping member (104e) is between 70 mm and 100 mm. Preferably, the length of the second clamping member (104e) is about 90 mm.

The distance between the first distal inwardly curved region (138e) and the second distal inwardly curved region (142e) is an offset distance of between 5 mm and 25 mm. Preferably, the offset distance is about 10 mm.

The first clamping member (102e) and the second clamping member (104e) fall within a maximum diameter of between 10 mm to 17 mm when the surgical clamp (100e) is in the closed configuration. The first clamping member (102e) and the second clamping member (104e) have a maximum diameter of 13 mm when the clamp is in the closed configuration. Preferably, the first clamping member (102e) and the second clamping member (104e) have a maximum diameter of 10 mm when the surgical clamp (100e) is in the closed configuration.

Each of the first clamping member (102e) and the second clamping member (104e) has a width in the range of 4 mm to 10 mm. Preferably, the first clamping member (102e) and the second clamping member (104e) has a width of about 7 mm. The thickness of each of the first clamping member (102e) and the second clamping member (104e) is in the range of 1 mm to 3 mm. Preferably, the thickness of each of the first clamping member (102e) and the second clamping member (104e) is about 2 mm.

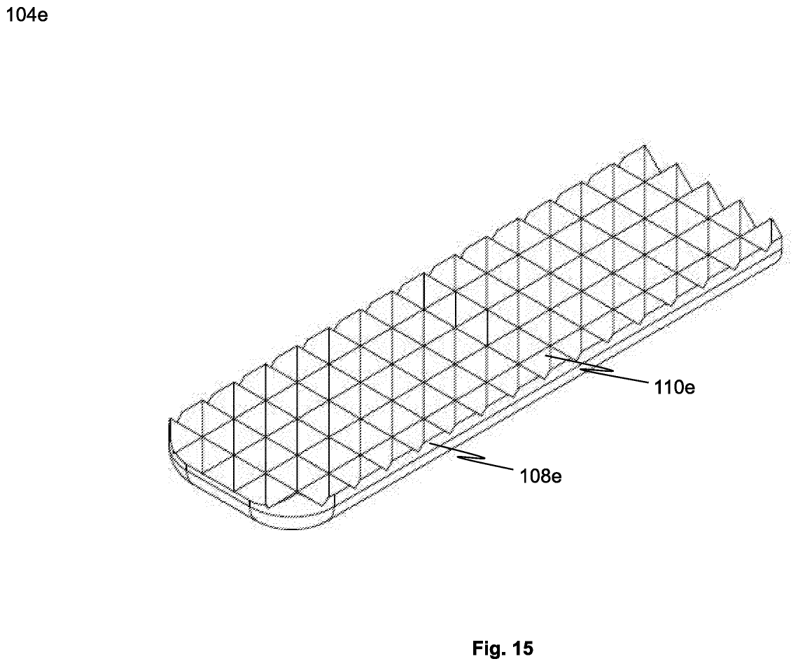

In the closed configuration of the surgical clamp (100e), the maximum gap defined between the two tissue engaging surfaces of the two tissue engaging portions (106e), (108e) of the respective clamping members (102e), (104e) is between 5 mm to 15 mm. Preferably, the maximum gap defined between the two tissue engaging surfaces is 13 mm. The arrangement of teeth (110e) (as shown in FIG. 15) of the two tissue engaging portions (106e), (108e) has the plurality of longitudinal rows of teeth in intersecting rows. A number of the plurality of longitudinal rows of teeth is between 2 and 5. Preferably, the number of the plurality of longitudinal rows of teeth is 4. Each tooth of the arrangement of teeth (110e) is substantially a 4-sided pyramid or a 3-sided pyramid in shape. Further, the arrangement of teeth (110e) may have any other type of suitable configuration. For example, in one embodiment the plurality of longitudinal rows of teeth are arranged continuously adjacent to each other, without any gaps. In another embodiment, each one of the plurality of longitudinal rows is spaced apart from another adjoining longitudinal row of teeth by a predetermined gap.

In the closed configuration, the surgical clamp (100e) has a maximum diameter of any cross-section of the surgical clamp (100e) in the range of 13 mm to 18 mm. The maximum diameter of any cross-section of the surgical clamp (100e) is about 13 mm.

The surgical clamp (100e) further includes an actuator (132e) which enables movement of the first clamping member (102e) and the second clamping member (104e) relative to each other between the open configuration and the closed configuration of the surgical clamp (100e) by driving manipulators (134e) of the actuator (132e). The manipulators (134e) may comprise a series of segments including sliding or jointed pieces and are pivotally connected to the first clamping member (102e) and the second clamping member (104e). Further, the actuator (132e) is a scissor-jack actuation arrangement in which the manipulators (134e) expand or contract in a manner resembling a pair of scissors upon clockwise or counter-clockwise rotation of the actuator (132e). This scissor jack has the smallest cross-sectional diameter in the closed configuration and can be opened sufficiently for the jaws to pass around the kidney without injuring the kidney. Alternatively, the actuating handles can be truncated to enable manipulation by the arms of a surgical robot, still with a scissor action at the pivot of the two elements of the clamp. Apposition of the actuating arms closes the tissue engaging portions of the clamp

Each of the manipulators (134e) has a length in the range of 50 mm to 100 mm. The length of each of the manipulators (134e) is preferably, 80 mm. Each of the manipulators (134e) may have a maximum length between 50 mm and 100 mm. Preferably, the maximum length is about 80 mm.

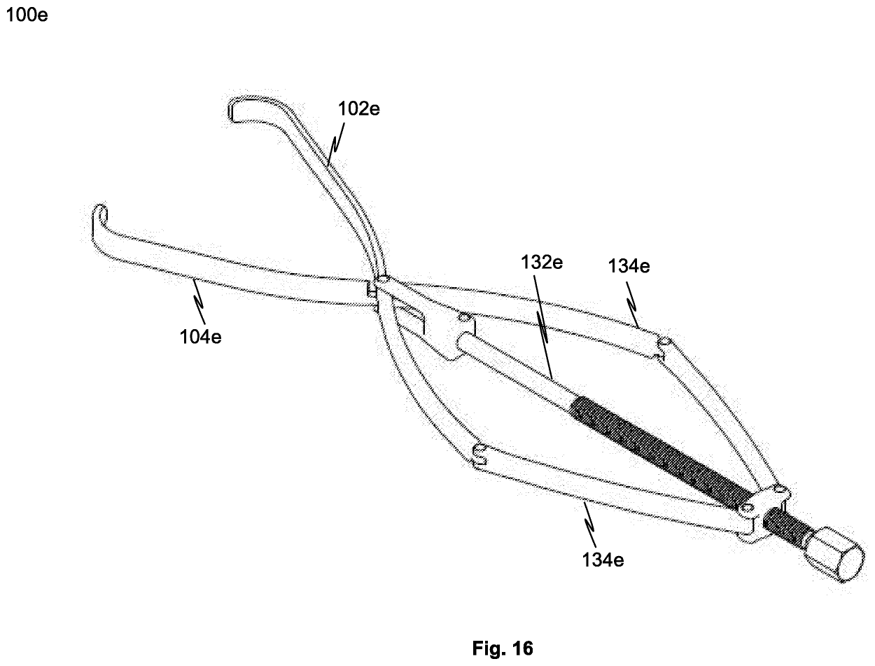

Referring to FIG. 16, the actuator (132e) is rotated in an anti-clockwise direction such that the manipulators (134e) move apart from each other. Since, the first clamping member (102e) and the second clamping member (104e) are connected pivotally with the manipulators (134e), the movement of the manipulators (134e) enables the first clamping member (102e) and the second clamping member (104e) to move away from each other for bringing the surgical clamp (100e) in the open configuration.

The surgical clamp (100e) may be used during a laparoscopic surgery, such as laparoscopic partial nephrectomy. The surgical clamp (100e) is used to clamp either end of the organ such as kidney to enable safe removal of tumours without cutting blood supply to the rest of the kidney and thereby damaging overall renal function for long term.

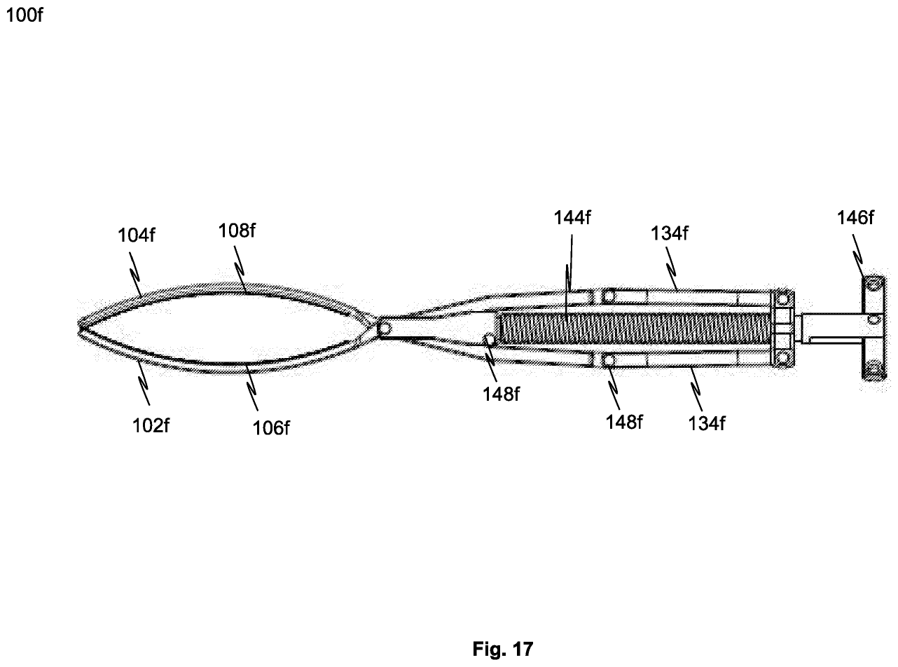

In the sixth embodiment of the invention, the surgical clamp (100f) as shown in FIG. 17, comprises a first clamping member (102f), a second clamping member (104f), a first tissue engaging portion (106f), a second tissue engaging portion (108f). Each of the first tissue engaging portion (106f) and the second tissue engaging portion (108f) comprise an arrangement of teeth (110f).

The structural arrangement of the first clamping member (102f), second clamping member (104f), first tissue engaging portion (106f), second tissue engaging portion (108f) and the arrangement of teeth (110f) in the sixth embodiment of the surgical clamp (100f) is the same as of the arrangement of these components in the first embodiment of the surgical clamp (100a).

Dimensions of these components of the surgical clamp (100f) are discussed below.

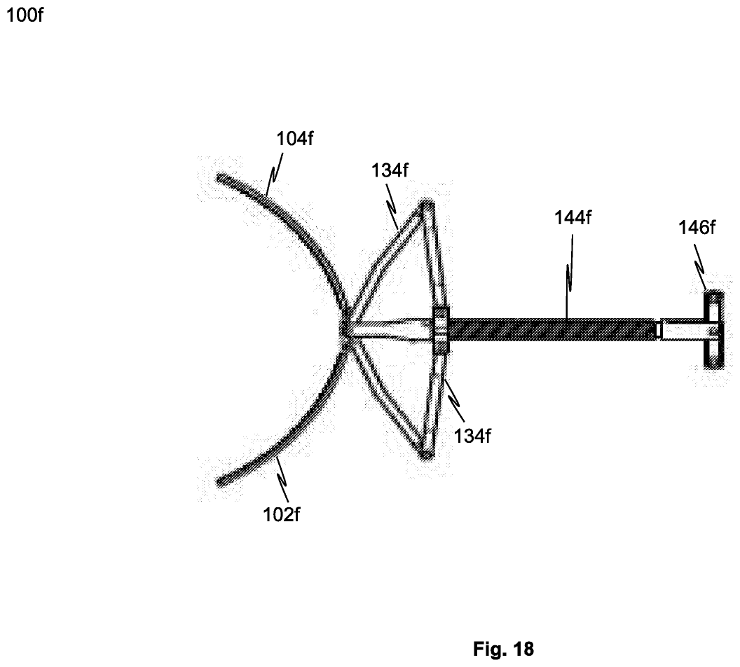

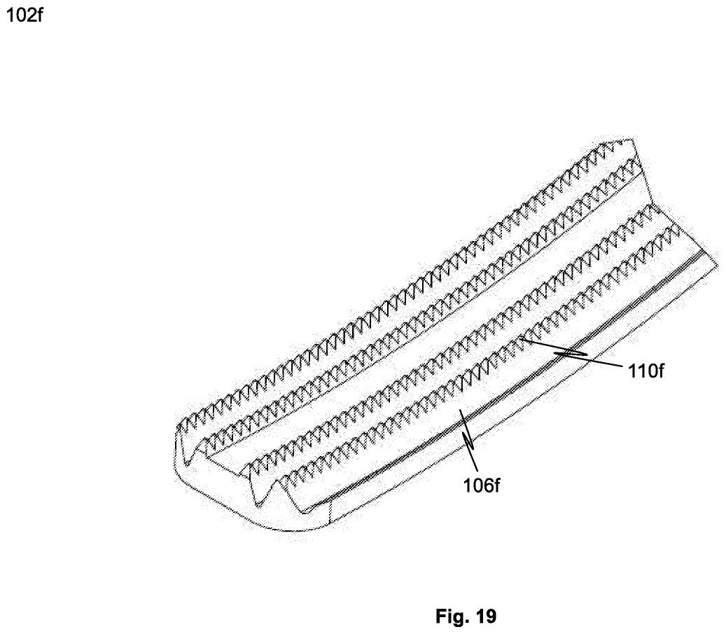

In the closed configuration of the surgical clamp (100f), the maximum gap defined between the two tissue engaging surfaces of the two tissue engaging portions (106f), (108f) of the respective clamping members (102f), (104f) is between 16 to 18 mm. The maximum gap defined between the two tissue engaging surfaces in the fully open configuration of the surgical clamp (100f) (as shown in FIG. 18), is 130 mm. The arrangement of teeth (110f) (as shown in FIG. 19), of the two tissue engaging portions (106f), (108f) has the plurality of longitudinal rows of teeth spaced from each other across the width of the two clamping members (102f), (104f). The plurality of longitudinal rows of teeth is between 2 and 20 rows of teeth. Preferably, the plurality of longitudinal rows of teeth is 4. Further, the teeth can be arranged in intersecting rows. Each tooth of the arrangement of teeth (110f) is substantially a 4-sided pyramid or a 3-sided pyramid in shape. Further, the arrangement of teeth (110f) may have any type of other suitable configuration.

In the closed configuration, the surgical clamp (100f) has a maximum diameter of 21 mm of any cross-section of the surgical clamp (100f). The overall length the surgical clamp (100f) is 214 mm.

The surgical clamp (100f) further includes a winding axle (144f), a lever (146f), rivets (148f) and manipulators (134f). The manipulators (134d) are pivotally connected to the first clamping member (102f) and the second clamping member (104f). The diameter of the pivot is in the range of 1 mm to 4 mm. Further, the length of the rivets (148f) is in the range of 7 mm to 11 mm. The structural and functional aspects of the winding axle (144f) and the manipulators (134f) in the sixth embodiment of the surgical clamp (100f) are same as of the arrangement and functional aspects of these components in the fourth embodiment of the surgical clamp (100d), wherein the winding axle (144f) resembles the actuator (132d) of the fourth embodiment of the surgical clamp (100d).

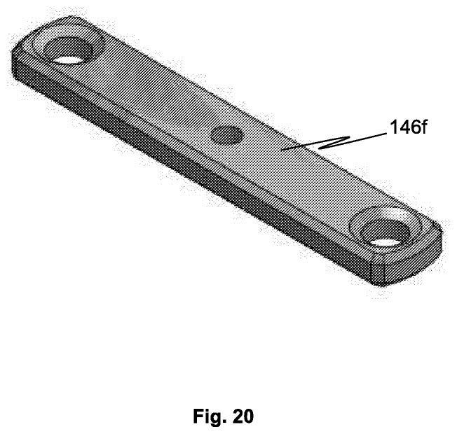

The winding axle (144f) has a width in the range of 7 mm to 12 mm. Further, the lever (146f) (as shown in FIG. 20) has a length in the range of 2 mm to 5 mm.

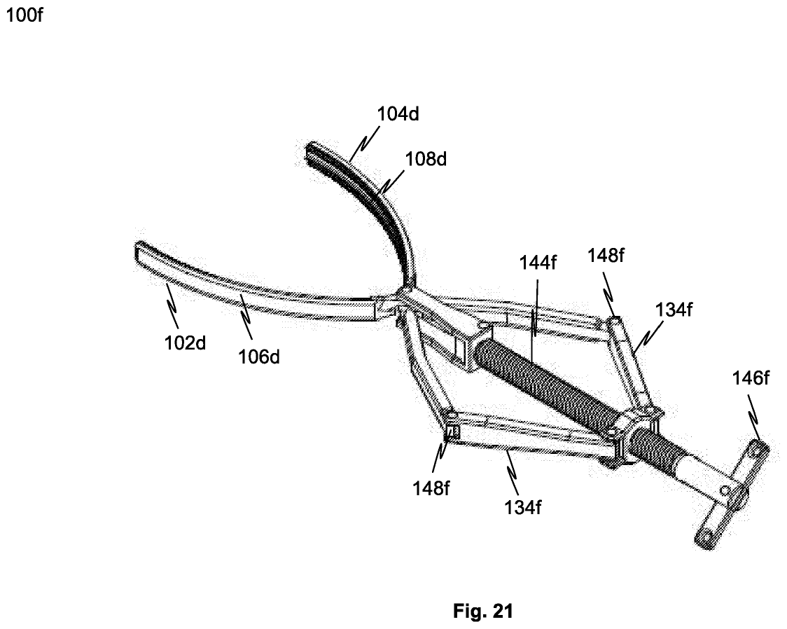

Referring to FIG. 21, the winding axle (144f) is rotated in an anti-clockwise direction such that the manipulators (134f) move apart from each other. Since, the first clamping member (102f) and the second clamping member (104f) are connected pivotally with the manipulators (134f), the movement of the manipulators (134f) enables the first clamping member (102f) and the second clamping member (104f) to move away from each other for bringing the surgical clamp (100f) in the open configuration.

The surgical clamp (100f) may be used during a laparoscopic surgery, such as laparoscopic partial nephrectomy. The surgical clamp (100f) is used to clamp either end of the organ such as kidney to enable safe removal of tumours without cutting blood supply to the rest of the kidney.

In another embodiment of the invention, the proximal ends of the first clamping member (102) and the second clamping member (104) are configured for connection to a surgical robot.

In another embodiment of the invention, the proximal ends of the first clamping member (102) and the second clamping member (104) are connected by a pivot mechanism. The pivot mechanism is an axle.

In another embodiment of the invention, the surgical clamp (100) includes a first and second lever member and wherein the first clamping member (102) is integral with the first lever member and the second clamping member (104) is integral with the second lever member such that relative movement of the first and second lever members towards each other causes the first clamping member (102) and the second clamping member (104) to move apart and relative movement of the first and second lever members away from each other causes the first clamping member (102) and the second clamping member (104) to move towards each other. The first lever member is a first handle (112) and the second lever member is a second handle (114).

In another embodiment of the invention, the surgical clamp (100) includes an actuator (132) for driving the first clamping member (102) and the second clamping member (104). The actuator (132) may be a scissor-jack actuation arrangement.

Each component of the invention may be made from any surgical grade, rigid material.

The teeth or arrangement of teeth (110) of any embodiment may be used in any other embodiment.

Advantages of the invention include easy clamping of the organs. The surgical clamp relieves the need to cool organs to preserve the activity or function of the organ without producing excessive bleeding and involves less dissection around the blood vessels, thereby shortening the operation. Embodiments of the surgical clamp enables removal of tumours from central position and/or from either ends of the organ. Further, the surgical clamp enables better suturing of the organ in less time as the organ can be positioned exactly where needed for access to suturing. The gap formed between the two tissue engaging surfaces of the surgical clamp facilitates only a small proportion of clamped tissue to experience the maximum compressive force of the surgical clamp as the gap provides relief from compression for a substantial proportion of the clamped tissue. In this way, the volume of tissue experiencing the maximum compressive force of the surgical clamp is relatively minimized. Also, the greater width of the two clamping members of the surgical clamp allows distribution of the compressive force of the clamping members across a larger surface area of tissue and thus reduces localized stress experienced by the tissue. In addition, the resilient and flexible nature of each clamping members attains the shape of the tissue and hence reduce the amount of compressive force transferred to the tissue.

### Interpretation

### Implementation:

It will be understood that the procedure of opening and closing the surgical clamp discussed is performed in one embodiment by an appropriate pivoting arrangement. It will also be understood that the invention is not limited to any position for placing the surgical clamp on the organ and that the invention may be implemented using any appropriate actuating mechanism for implementing the opening and closing and thus placement of the surgical clamp described herein.

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description of specific Embodiments are hereby expressly incorporated into this detailed description of specific embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose.

For the purposes of this specification, the term “surgical clamp” shall be construed to mean a clamp used to hold the organ in a particular position to allow surgeons to access and operate on surrounding tissues with interruption to the blood flow beyond the clamp.

As used herein the term “and/or” means “and” or “or”, or both.

As used herein “(s)” following a noun means the plural and/or singular forms of the noun.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, placement of the surgical clamp on kidney for partial nephrectomy of kidney cancer given above is merely representative of procedure that may be used to remove tumours from any organ or tissue or blood vessel. Positions of clamping the surgical clamp may be changed to the positions described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the surgical equipment industries or medical device industries.

1-71. (canceled)

72. A surgical clamp for clamping a kidney, said clamp comprising first and second clamping members each having a distal end and a proximal end and being pivotably connected to each other at their respective proximal ends so as to be movable between an open configuration and a closed configuration of said clamp, each of said clamping members being opposed, and said first and second clamping members having a corresponding first and second tissue engaging portions each comprising a strip arrangement of teeth, wherein in said closed configuration only the distal ends of said first and second clamping members contact each other; and each of said first and second clamping members are generally concave such that in said closed configuration said first and second tissue engaging portions are separated by a gap there between which extends between said proximal and distal ends, said gap and the width of said teeth strip arrangement being dimensioned to apply a maximum compressive force to only a small proportion of the clamped kidney.

73. The surgical clamp as claimed in claim 72 wherein the gap between said first and second tissue engaging portions when said clamping members are in said closed configuration is within the range of 5-29 mm.

74. The surgical clamp as claimed in claim 72 wherein the width of each of the first and second clamping members is within the range of 4-25 mm.

75. The surgical clamp as claimed in claim 72 wherein said concave tissue engaging portions each comprise a first arc having a radius of curvature within the range of 90-210 mm.

76. The surgical clamp as claimed in claim 72 wherein the length of each of said first and second clamping members is within the range of 60-120 mm.

77. The surgical clamp as claimed in claim 72 wherein the thickness of each of the first and second clamping members is within the range of from 1-4 mm.

78. The surgical clamp as claimed claim 72 wherein the pivotal connection between said first and second clamping members is selected from the group of scissors and scissors-jack actuation arrangements.

79. The surgical clamp as claimed claim 72 wherein when viewed so that said first clamping member overlies said second clamping member, both said clamping members appear to be straight.

80. The surgical clamp as claimed in claim 79 wherein one of said clamping members is longer than the other and both are hooked at their distal ends.

81. The surgical clamp as claimed in claim 72 wherein when viewed so that said first clamping member overlies said second clamping member, both said clamping members appeared to be curved.

82. The surgical clamp as claimed in claim 72 wherein the gap between said first and second tissue engaging portions when said clamping members are in said closed configuration is within the range of 7-16 mm.

83. The surgical clamp as claimed in claim 72 wherein the width of each of the first and second clamping members is within the range of 7-12 mm.

84. The surgical clamp as claimed in claim 72 wherein said concave tissue engaging portions each comprise a first arc having a radius of curvature within the range of 104-184 mm.

85. The surgical clamp as claimed in claim 72 wherein the length of each of said first and second clamping members is within the range of 80-100 mm.
